(19) United States
(12) Patent Application Publication
(10) Pub. No.: US 2017/0037348 A1
(43) Pub. Date: Feb. 9, 2017

(54) GRAVITY FLOW TUBULAR PHOTOBIOREACTOR AND PHOTOBIOREACTOR FARM

(71) Applicant: Advanced Algae, Inc., Wilmington, CA (US)

(72) Inventor: Dale Hinkens, Wilmington, CA (US)

(21) Appl. No.: 15/064,541

(22) Filed: Mar. 8, 2016

Related U.S. Application Data
(63) Continuation of application No. 14/163,974, filed on Jan. 24, 2014, now abandoned.
Application No. 14/163,974 is a continuation of application No. 13/390,340, filed on Feb. 14, 2012, now abandoned.
(60) Provisional application No. 61/274,449, filed on Aug. 17, 2009.

(51) Int. Cl. C12M 1/00; C12M 3/00; C12M 1/12

(52) U.S. Cl. CPC C12M 21/02; C12M 23/06; C12M 23/22; C12M 23/26; C12M 23/48; C12M 29/24

(57) ABSTRACT

A gravity flow photobioreactor core (10) comprised of a support means (3); a tube (5) that continuously runs and curls with declination about a vertical axis to form a stack (7) of levels (9) and having an inlet sparge (11); a gas exchange tank (13) and a central feed pipe (15) with a sparge (17). A gravity flow photobioreactor farm comprised of a bottom tank (19); a pump (21); a plurality of bioreactor cores (10) connected in series at decreasing elevations and a return pipe (23).

### CROSS-REFERENCE TO RELATED APPLICATIONS

This application is a continuation of U.S. application Ser. No. 14/163,974, filed Jan. 24, 2014, which is a continuation of U.S. application Ser. No. 13/390,340, filed Feb. 14, 2012, which is the U.S. National Phase Application under 35 U.S.C. §371 of International Application PCT/US2010/045687, filed on Aug. 17, 2010, which claims the benefit of U.S. Provisional Application Ser. No. 61/274,449, filed on Aug. 17, 2009. The entire contents of each of which are hereby incorporated by reference.

### FIELD OF THE INVENTION

This invention pertains generally to bioreactors and more particularly to tubular-type photobioreactors.

### BACKGROUND OF THE INVENTION

The world has entered an era of climate shifts for which there is evidence that this attributable to carbon dioxide from the burning of fossil fuels. Concomitantly, the world-wide supply of fossil fuels is being exhausted. In addition, nitrogen oxides (NOx) are being admitted into the air at levels for which there is evidence that this is causing health abnormalities and shortening life spans. This in turn is imposing a financial burden on the healthcare and insurance system.

Governments around the world are responding by regulations that limit the emission of carbon dioxide and nitrogen oxide and/or by imposing financial penalties on the emission of carbon dioxide and nitrogen oxide. Socially conscious activists and governments are promoting environmentally friendly technology that is going by the colloquial phrase “green technology,” including photobioreactors.

Australian patent publication number 2006100045 is known in the art of photobioreactors. This patent publication relates to a photobioreactor for the cultivation and harvesting of a blue-green algae solution. The photobioreactor design of the invention consists of the following components. A vertical coil of transparent or semi-transparent tubing joined at top and bottom via a tube or tank so as to provide a system through which a solution of blue-green algae, water, nutrients and gas can circulate. The coil may be made into shape other than a cylinder, such as a cone, oval cylinder, cuboid, tetrahedron, pyramid or a flat horizontal coil shape. A tap at the base of the photobioreactor to allow the solution to be drained off and harvested or cleaning of the photobioreactor. A gas inlet (11) into the tubing, connected at the base of the coil, above the tap so that gas rises up through the solution in the tubular coil, causing it to circulate. A gas outlet at the uppermost point of the photobioreactor. This invention has the disadvantage of being inefficient, building up oxygen that retards algae growth, not having a significant sequestration capability and not teaching a multireactor system that is mechanically simple and energy efficient.

Japanese patent publication number 91-21835 is also known in the art. This patent publication provides a tubular-type photobioreactor designed with a light transmissive tube installed spirally and spacedly on the side of a conical body to effect greater light receiving area despite small installation area. The photobioreactor is designed to culture for example fine algae. This invention has the disadvantage building up oxygen that retards algae growth, not having a significant sequestration capability and not teaching a multireactor system that is mechanically simple and energy efficient.

World Intellectual Property Organization patent publication WO 9928018 (A1) relates to a method and device for reducing the concentration of ingredients in a gas and in a liquid. According to the inventive method, the liquid is first guided through a washing unit. A gas containing ingredients is guided into the washing unit and comes in contact with the liquid in the washing unit such that the liquid absorbs ingredients in an optionally converted form from the gas. Afterwards, the gas whose ingredients have been reduced is removed from the washing unit. The liquid enriched with ingredients is at least partially guided from the washing unit to a conversion device containing microalgae in which the ingredients are at least partially absorbed by the migroalgae by means of photosynthetic activation, and the microalgae are at least partially separated from the liquid after they have absorbed ingredients. This invention has the disadvantage utilizing a prewashing unit, not having a significant sequestration capability and not teaching a multireactor system that is mechanically simple and energy efficient.

Accordingly, there exists a need for a bioreactor with enhanced oxygen exchange that does not employ sprayers, is mechanically simple and energy efficient.

There is a need for a bioreactor that significantly sequesters carbon dioxide.

There is a need for a bioreactor that significantly sequesters nitrogen oxides.

There is a need for a bioreactor that quickly produces significant quantities of algae or other microorganism that is usable as a feedstock for the production of biofuel and biomass.

There is a need for a multi-bioreactor system that does not require a pre-washing unit.

There is a need for a multi-bioreactor system that moves material in a manner that is mechanically simple and energy efficient.

The present invention satisfies these needs, as well as others, and generally overcomes the presently known deficiencies in the art.

### SUMMARY OF THE INVENTION

The present invention is directed to, inter alia, a bioreactor for growing a microorganism (especially algae,) a series of bioreactor cores that are joined together in a farm, a method for the sequestration of carbon dioxide, a method for the sequestration of nitrogen oxides, a method for the collection of oxygen, and method for the production of a biofuel feedstock.

An object of the present invention is a bioreactor with enhanced oxygen exchange that does not employ a sprayer, is mechanically simple, and energy efficient.

Another object of the present invention is a bioreactor and muti-bioreactor system that significantly sequesters carbon dioxide.

Another object of the present invention is a bioreactor and muti-bioreactor system that significantly sequesters nitrogen oxides.

Another object of the present invention is a muti-bioreactor system that does not require a pre-washing unit.

Another object of the present invention is a bioreactor and muti-bioreactor system that moves material in a manner that is mechanically simple and energy efficient.

Another object of the present invention is a muti-bioreactor system that employs gravity to move material/slurry so as to reduce the utilization of pumps, motors and compressed air to do the same.

Concomitant objects of the invention are a bioreactor and a multi-bioreactor system that consumes less energy, is less expensive and less subject to breaking with the incursion of downtime and repair cost.

Another object of the present invention is to collect diatomic oxygen for use in aiding combustion.

Another object of the present invention is to produce a feedstock for biofuel and biomass.

One aspect present invention is a bioreactor. The bioreactor has a support means having vertical height. Mounted to this support means is a tube that at a minimum partially passes light through itself This tube starts at an upper position, continuously runs and curls with declination about a vertical axis to form a stack of levels. Each level encompassing about 360 degrees around the vertical axis. The radial distance between the tube and the vertical axis indexes within the stack so as to enhance the tube's exposure to light emanating from above the stack relative to the tube being vertically aligned at a constant radial distance from the axis within the stack. The tube ends in lower position. There is a sparge for introducing a froth of gas, usually carbon dioxide and/or nitrogen oxides, into the tube. Above the tube and mounted to the support means is a gas exchange tank. This tank empties by gravity into the upper end of the tube. This gas exchange tank has a slurry entry inlet and an outlet for the elimination of gas.

There is a bottom tank that is a reservoir for a microorganism, for example algae, nutrients and water. A pump is connected to the bottom tank and to a central feed pipe. The central feed pipe runs from the pump to the slurry entry inlet of the gas exchange tank. The central feed pipe has a sparge for introducing a froth of gas into the central feed pipe. There is a return pipe that runs from the lower end of the tube to the bottom tank. The bioreactor is a closed system where the entry and release of fluid and gas is controlled.

Another aspect of the present invention is a support means for the bioreactor as just described. The support means has an upper frame, a lower frame, and vertical supports that run from the lower frame to the upper frame. A plurality of cables depend from the upper frame and attach to the tube so as to support the tube in the stack. There is a column onto which is mounted the gas exchange tank in a position generally above the stack.

Another aspect of the present invention is a bioreactor farm comprised of a successive series of bioreactor cores on a surface. There is a first bioreactor core along the lines of that which was just described. There is a bottom tank, and a pump where the inlet side of the pump is in fluid communication with the bottom tank. The outlet side of the pump is in fluid communication with the central feed pipe of the first bioreactor core. There is a subseries of bioreactor cores where the lower opening of the tube of preceding bioreactor core is in fluid communication with the central feed pipe of succeeding bioreactor core. These bioreactor cores rest on the surface such that succeeding bioreactor cores decrease in elevation relative to the preceding bioreactor core. Accordingly, the fluid fill level of a gas exchange tank in a succeeding bioreactor core is generally lower than the bottom of the gas exchange tank of a preceding bioreactor. There is a final bioreactor core. A return pipe runs from the lower opening of the tube of the final bioreactor to the bottom tank. The bioreactor farm is a closed system where the entry and release of fluid and gas is controlled.

Another aspect of the present invention is method for sequestration of carbon dioxide. The method is comprised of steps. The steps are to provide a bioreactor farm as just described; introduce into the bioreactor cores of the farm a mixture of a microorganism that metabolizes carbon dioxide, nutrients and water; introduce carbon dioxide into the sparge for introducing a gas in communication with the tube of at least one bioreactor core and actuation of the pump (21).

Another aspect of the present invention is method for sequestration of nitrogen oxides. The method is comprised of steps. The steps are to provide a bioreactor farm as just described; introduce into the bioreactor cores of the farm a mixture of a microorganism that metabolizes nitrogen oxides, nutrients and water; introduce nitrogen oxides into the sparge for introducing a gas in communication with the tube of at least one bioreactor core and actuation of the pump (21).

The previously described versions of the present invention has many advantages which include low energy consumption, durability arising from utilization of gravity to move material, a high removal of oxygen which impedes the growth of algae, fast and abundant algae growth, the sequestration of nitrogen oxides, the sequestration of carbon dioxide, and the production of a feedstock for biofuel.

### BRIEF DESCRIPTION OF THE DRAWINGS

These and other features, aspects and advantages of the present invention will become better understood with reference to the following description, appended claims and accompanying drawings where:

FIG. 1 is a perspective view of a bioreactor farm according to the present invention;

FIG. 2 is a perspective view of a bioreactor according to the present invention;

FIG. 3 is a perspective view of a gas exchange tank (13) and center feed pipe according to the present invention;

FIG. 4 is a side plan view of a support means for a bioreactor according to the present invention;

FIG. 5 is an enlarged view of a diagonal support and arms of the support means of FIG. 4;

FIG. 6 is a diagrammatic view of a bioreactor according to the present invention;

FIG. 7 is a perspective view of a support means for a plurality of bioreactors in a farm according to the present invention;

FIG. 8 is a top plan view of a cage of the support means of FIG. 7;

FIG. 9 is a perspective view of a column and gas exchange tank engaging an upper support of the support means of FIG. 7;

FIG. 10 is a perspective view of a column and gas exchange tank (13), along with vertical support ribs, engaging an upper support of the support means of FIG. 7;

FIG. 11A is a perspective view of a gas elimination outlet and manifold in connection with a gas exchange tank and FIG. 11B is a perspective view of a center feed pipe in connection with a gas exchange tank and

FIG. 12 is a diagrammatic view of a bioreactor farm according to the present invention.

### DETAILED DESCRIPTION OF THE PREFERRED EMBODIMENT

The present invention is described more fully in the following disclosure. In this disclosure, there is a discussion of embodiments of the invention and references to the accompanying drawings in which embodiments of the invention are shown. These specific embodiments are provided so that this invention will be understood by those skilled in the art. This invention is not limited to the specific embodiments set forth herein below and in the drawings. The invention is embodied in many different forms and should be construed as such with reference to the appended claims.

The invention pertains, inter alia, to a bio reactor for growing a microorganism, especially algae, a bioreactor farm of joined bioreactor cores (10), a method for the sequestration of carbon dioxide, a method for the sequestration of nitrogen oxides, a method for the collection of oxygen and method for the production of a biofuel feedstock.

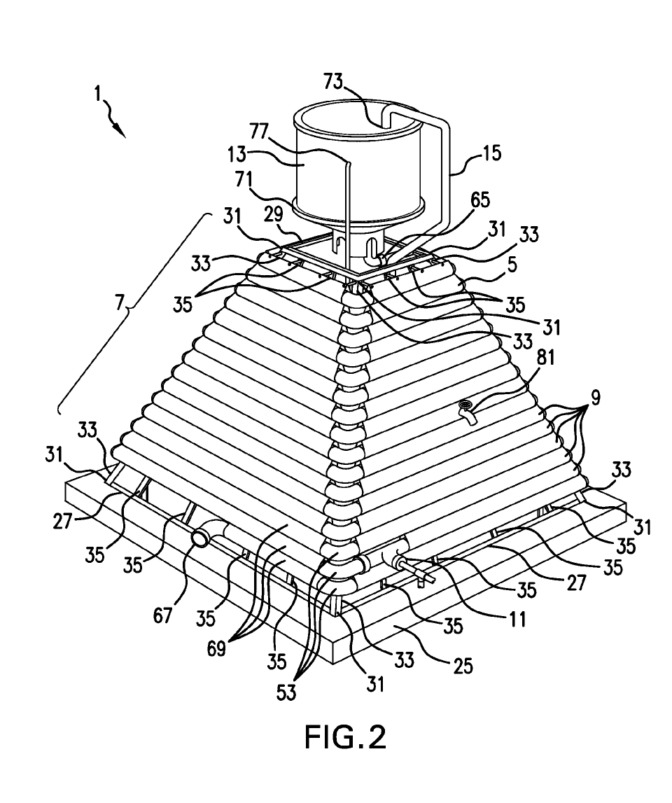

Referring to FIG. 2, in general terms and for an overview, the major components and assemblies of a bioreactor (1) are a support means (3); a tube (5) that continuously runs and curls with declination about a vertical axis to form a stack (7) of levels (9) and has a inlet/sparge (11) for an effluent; a gas exchange tank (13); a central feed pipe (15) which optionally has a sparge (17); a settling tank (19); a pump (21) and a return pipe (23). In the discussion that follows, each of these major components and assemblies is discussed, along with other structures and components in the embodiments of this invention. Thereafter, there is a discussion on the methods and the use of the invention.

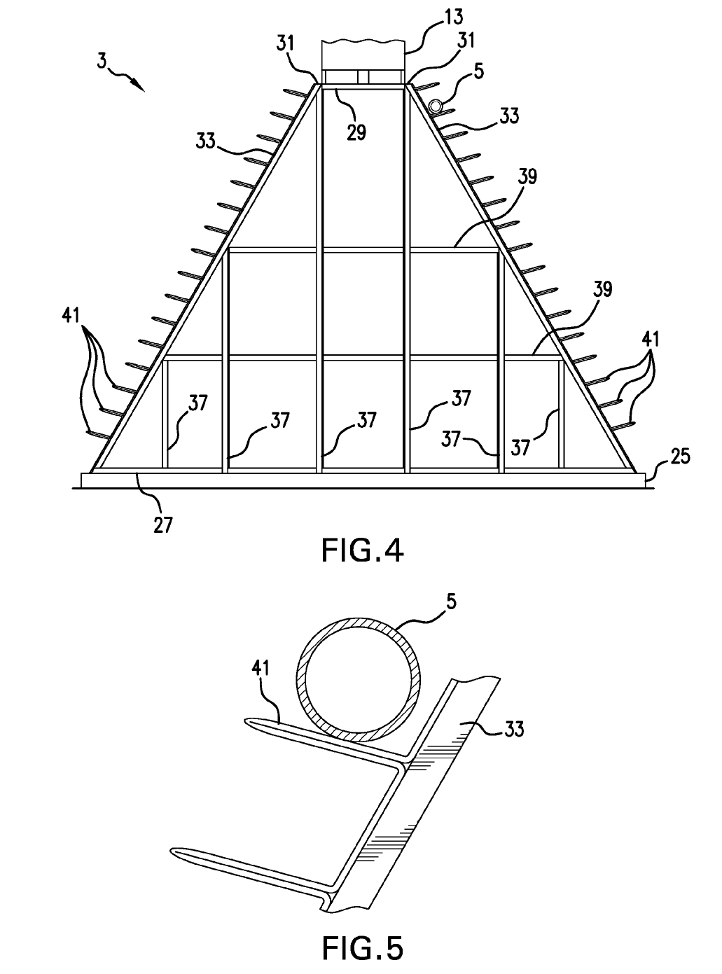

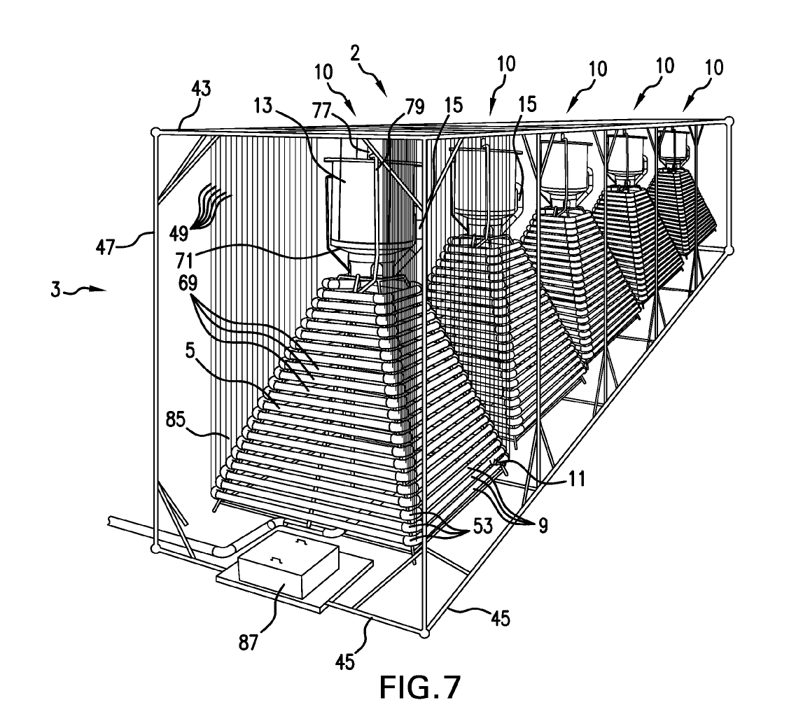

Referring to FIGS. 4 and 7, the support means (3) is characterized by having vertical height. The vertical height of the support means (3) is usually up to about 25 feet. The support means (3) provides support, inter alia, for the tube (5) and for a gas exchange tank (13). The support means (3) in turn rests and/or is supported by a base means (25) (discussed below) which interfaces with a surface. The support means (3) is preferably an open air-like structure without panels and walls such that light can substantially pass through it. This facilitates the tube (5) being exposed to light from all directions and loosely referred to as 360° exposure.

One structure for the support means (3) is a Christmas tree-like structure (not illustrated.) This structure has a central support column, typically made of metal, around which numerous branches are attached in layers. The numerous branches circle the column in layers with the shortest branches being on top and the longest branches being on the bottom. The structure is that of a large cone or a Christmas tree. This structure can be set in a square or rectangular base to keep the support steady.

Referring to FIGS. 2 and 4, another structure for the support means (3) is a truncated pyramid or truncated tetrahedron-like structure. This structure is comprised of a lower square-like frame (27) having vertices and an upper square-like frame (29) having vertices (31). The upper square-like frame 29 is smaller than the lower square-like frame (29). The term “square-like” implies that frame approximates a parallelogram and need not have precisely four sides, straight sides, equal length sides and/or 90 degree angles. The suffix “-like” as used herein has this meaning of generally approximating a shape. The upper square-like frame (29) and lower square-like frame (27) are approximately centered on a vertical axis. There are four main diagonal support members (33) that are each attached to vertices (31) of the upper square-like frame (29) and the lower square-like frame (27) so as to form a configuration that has a truncated pyramid-like shape.

Optionally, there can be intermediate diagonal support members (35) that are each attached the upper square-like frame (29) and lower square-like frame (27); vertical support members (37) that run from a base means (25) (discussed below) or surface to a main diagonal support member (33) and horizontal support members (39). The support members (33, 35, 37 and 39) can be angle iron, steel I-beam, metal bars, pipes or greenhouse frame and can be welded together and/or joined with brackets and screws.

Referring to FIGS. 4 and 5, there is a flat bar strip (41) mounted along the main diagonals with a plurality of bends that bend back on themselves to form shelves for supporting the tube (5) in the levels (9) of the stack (7). Preferably, the shelves are on an angle so that the tube (5) can be straddled between the shelve and the main diagonal support (33).

Referring to FIG. 7, another physical structure, and a most preferred physical structure is a comprised of a upper frame (43), typically a square or a rectangle, that encompasses a bioreactor (1) or a series of bioreactor cores (10) in a bioreactor farm (2) (discussed below.) The shape is not critical and other shapes can be deployed. Optionally, there is a lower frame (45) complimentary to the upper frame (43). There are vertical supports (47) that run from lower frame (45) or base means (25) to the upper frame (43) to support the upper frame (43) at a position above the tube (5).

Continuing to refer to FIG. 7, there is plurality of cables (49) that drop or depend from the upper frame (43) to support the tube (5). Thus, this structure provides “top support” for the tube (5). Referring to FIGS. 9 and 10, there is a column (51) to support a gas exchange tank (13). This column is typically positioned in the center of the bioreactor (1) or bioreactor cores (10). Where the bioreactor (1) or bioreactor cores (10) is a pyramid-like in shape, the column (51) is in the center of the pyramid. The column (51) is sufficiently strong to support a gas exchange tank (13) having a weight of 8,000 pounds. There may be depending cables (49) from the upper frame (43) to gas exchange tank (13) to stabilize the gas exchange tank (13).

Notwithstanding, the weight of the gas exchange tank (13) is borne by the columns (51) and is off the upper frame (43). There may be a plate extending between the column (51) and the tube (5) to stabilize the tube (5).

Referring to FIGS. 7 and 9, the column (51) and frames (43 and 45) are typically made from I-beams or greenhouse frame which are welded and/or joined with brackets and screws. The cables (49) are typically steel cable. There can be precision drilled holes in the I-beams by which to fasten precisely measured cables that drop down to attach to stainless bands around each elbow (53) (discussed below.) The stainless steel bands have eyes hook for attaching to the cables. In this structure there is no need for an underneath support structure for the pipes (discussed below.) Referring to FIG. 10, in a preferred embodiment, there are vertical ribs (55) that extend from the column (51) to support the upper frame (43). This reduces the strength of the material and construction required for the upper frame (43).

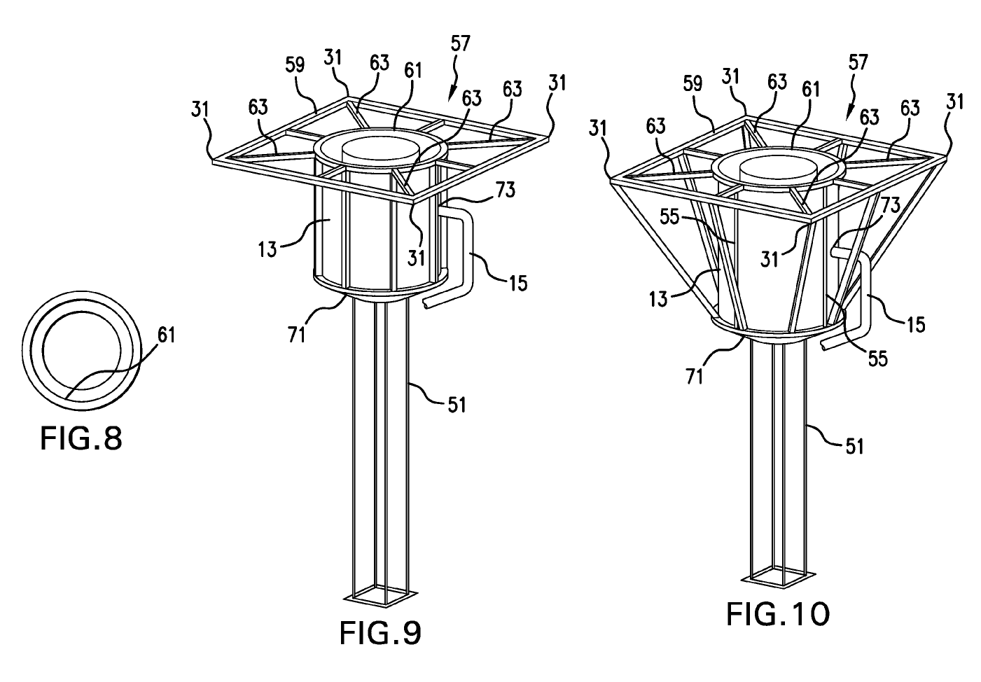

Referring to FIGS. 8 and 9, in a more preferred embodiment, there is a cage (55) that is capable of receiving the gas exchange tank (13) that extends between the column (51) and the upper frame (43) so as to provide vertical support to the upper frame (43). The cage (55) is comprised of vertical support members (59), horizontal support members (63); which can be on diagonal with respect to the upper frame (43) and a circular inset (61). The horizontal support members (63) run from the upper frame (43) to the circular inset (61). The vertical support members (59) run from the column (51) and the circular inset (61). More preferably, cables (49) depend from the horizontal support members (63) which are on diagonal and positioned above the elbow tubes of a truncated pyramid shaped tube (5).

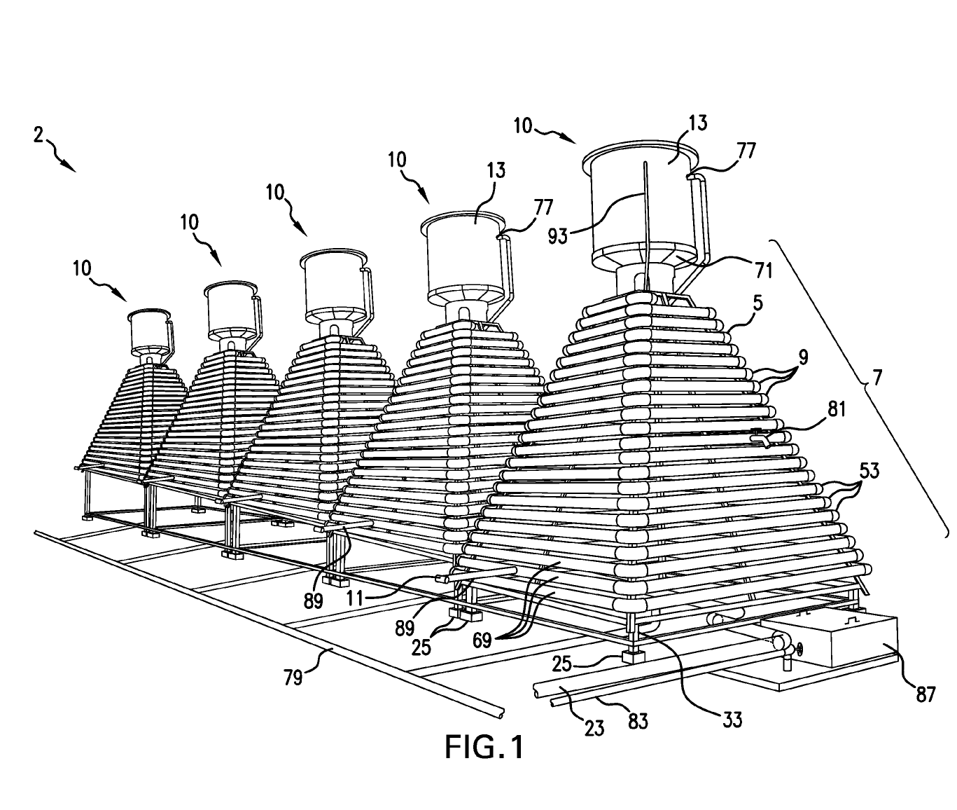

Referring to FIGS. 1 and 2, the base means (25) is the ground, a surface, a slab, a plurality of pads or a plurality of pylons.

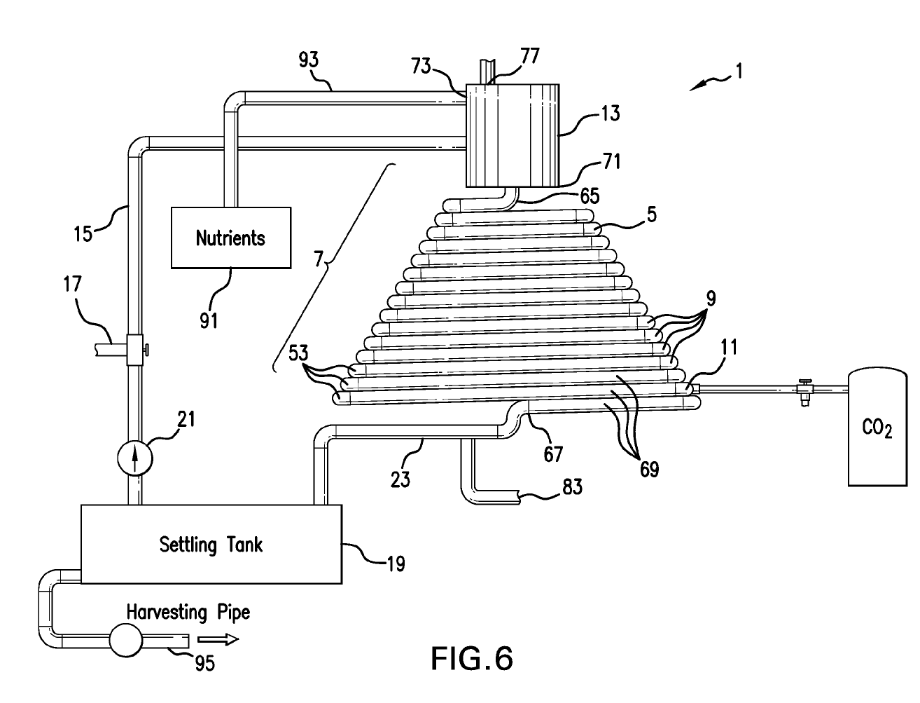

Referring to FIGS. 2 and 6, the tube (5) is an elongated conduit having an upper opening (65) and lower opening (67). At a minimum, the tube (5) partially passes light there through. More preferably, the tube (5) is substantially transparent and most preferably, it is transparent. The tube (5) has flexible or rigid walls and preferably the tube (5) is annular and with rigid walls. The internal diameter of the tube (5) ranges from about one inch to about twelve inches. Preferably, the tube (5) has an internal diameter between about 3 inches to about 5 inches. Most preferably, the internal diameter of the tube (5) is about 4 inches. The wall thickness is sufficiently great to withstand the pressure of the system's fluid contents. The material of the tube (5) is non-toxic to microorganisms, especially algae. Preferred materials are transparent plastics. More preferred materials are polyvinyl chloride (PVC), acrylic and polycarbonate. A most preferred material is polycarbonate.

Continuing to referring to FIGS. 2 and 6, the tube (5) spirals, curls and bends with declination around a vertical axis to form a stack (7) of levels (9). At about a minimum, the declination is such that tube (5) loses approximately two (2) inches in elevation with each level (9). This enables a desired downward flow of liquid under the force of gravity. More preferably, the slope of declination lowers each level between about 4 inches to about 8 inches. Most preferably, the slope of declination lowers each level about 6 inches.

Continuing to refer to FIGS. 2 and 6, preferably, the levels (9) are in substantially parallel planes. The spacing of the tube (5) from each other in the vertical direction in going from level (9) to level (9) is preferably such that the tube (5) can be efficiently exposed to sunlight and is not so great so as to waste space. More preferably, the spacing between levels is between about one inch to about three inches with two inches most preferred.

Continuing to refer to FIGS. 2 and 6, in preferred embodiments, as the tube (5) spirals, curls, bends and runs downward, it gets sequentially larger in encompassed surface area. More preferably, the tube (5) in a particular level (9) indexes out from the vertical axis by the diameter of the tube relative to the above level (9) so that the tube (5) is not in vertical alignment in going from level to level (9). This maximizes exposure of the tube (5) to sunlight.

In more preferred embodiments, the levels (9) of tube (5) in the stack (7) are parallelogram-like or square-like in shape. Parrallogram-like means that frame approximates a parallelogram and need not have precisely four sides, straight sides, equal length sides and/or 90 degree angles.

Accordingly, the stack (7) has a pyramid or tetrahedron-like shape. In these embodiment, the tube (5) can be constructed from a kit comprised of straight lengths (69) and approximately 90° elbow tubes (53). The elbow tubes (53) are made from a material that is non-toxic to microorganisms, especially algae, and preferably, from polyvinyl chloride (PVC), acrylic or polycarbonate. A most preferred material is PVC. A fluid tight attachment of the straight lengths (69) to elbow tubes (53) can be achieved by dipping the end of a straight length of tube (5) in an adhesive material and then placing the elbow tube (53) on the end.

Preferably, the levels (9) of the tube (5) in the stack (7) encompass an area ranging from about four (4) square feet at the top level to about 625 square feet at the bottom level. More preferably, the levels encompassing an area ranging from between about 9 square feet to about 169 square feet. Most preferably, the bottom level encompasses a surface area of about 100 square feet. Preferably, the stack (7) has a vertical height between of about seven feet to about eleven feet with nine feet most preferred.

Referring to FIGS. 1, 2 and 6, there is a sparge or effluent inlet (11) for introducing a gas in communication with the tube (5) in the stack (7). The function of the sparge or effluent inlet (11) is to introduce carbon dioxide, nitrogen oxides and other gasses and liquids into the bioreactor to be metabolized by the microorganism, especially algae, that is resident in the bioreactor. The bubbles introduce carbon dioxide, nitrogen oxides and other gasses are buoyant and travel upwards and counter current to a slurry in the tube (5) which is flowing by gravity downward. Typically, this sparge or effluent inlet (11) is at a lower position within the stack and most preferably, it is positioned at the second lowest level (9). The lower the position in the stack for the sparge or effluent inlet, the greater the residency time of carbon dioxide and nitrogen oxides in the tube (5). A fifteen minute residency time is achievable.

In a preferred embodiment, the sparge (11) introduces the carbon dioxide, nitrogen oxide and/or other gasses as a robust froth of microbubbles having significant surface area to facilitate the gas dissolving in a slurry in the tube (5). In a more preferred embodiment, the sparge has sintered stainless steel or air stone porous element and in a most preferred embodiment, the porous element is sintered stainless steel. Preferably, the porous element has a wide pore size so as to facilitate the entry of gas a low pressure between about six to about ten pounds per square inch.

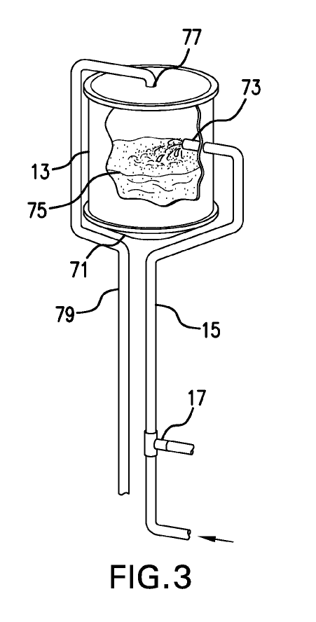

Referring to FIG. 3, the gas exchange tank (13) is closed vessel with defined inlets and outlets. Thus, pressure can build up in the gas exchange tank (13). The gas exchange tank (13) has a capacity of at least about 350 gallons and preferably between 375 to 400 gallons. Preferably the gas exchange tank (13) has a height between about four feet to about six feet with five feet most preferred. This provides about 1,200 pounds of gravity induced hydraulic force to push slurry into the next bioreactor core (10) of a bioreactor farm (2) (discussed below.) The gas exchange tank (13) is mounted to and supported by the support means (3) at a position that is generally above the stack (7).

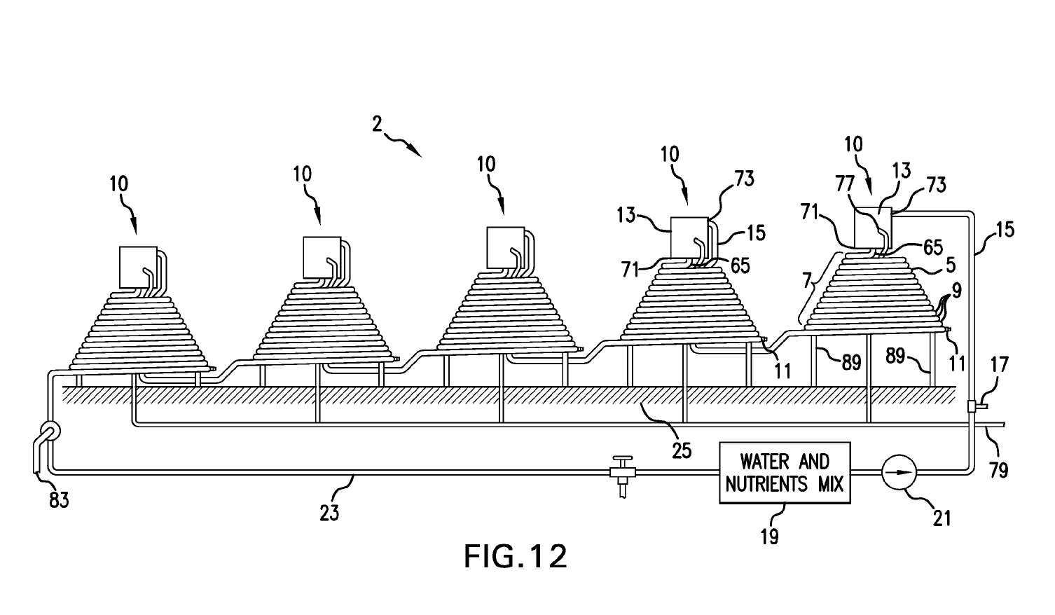

Continuing to refer to FIG. 3, the gas exchange tank (13) has a bottom (71) and this bottom (71) can be flat, conical or other shape. A conical bottom is preferred to impede the settling of algae or other microorganism. Referring to FIGS. 6 and 12, at the bottom portion of the gas exchange tank (13) is an outlet along with piping to connect it to the upper opening (65) of the tube (5). During operation of the bioreactor (1) or bioreactor core (10), slurry flows from the gas exchange tank (13) to the tube (5).

Continuing to refer to FIG. 3, the gas exchange tank (13) has a slurry entry inlet (73). From this slurry entry inlet (73) there is piping to connect to a central feed pipe (15) (discussed below.) During operation of the bioreactor (1) or bioreactor core (10) slurry flows up the central feed pipe (15) and into the gas exchange tank (13). Optionally, the gas exchange tank (13) can have a fluid fill level (75). This is a level in the gas exchange tank (13) at which fluid does generally rise above during the operation of the bioreactor (1). In a preferred embodiment, slurry entry inlet (73) is above the fluid fill level (75) or off of the top of the gas exchange tank (13). A slurry entry inlet (73) on the side of the side of the gas exchange tank (13) is referred so as not to increase to overall height of the bioreactor (1).

Accordingly, during operation of the bioreactor (1), as slurry exits the slurry entry inlet (73), it splashes down into a reservoir of slurry in the bottom of the gas exchange tank (13). This splashing creates a froth and otherwise enhances the release of gas, especially diatomic oxygen, from the slurry. In a most preferred embodiment, slurry pulsates (that is, the flow rate ebbs up and down) to increase the splashing and hence the freeing of gas for discharge out of the gas exchange tank (13).

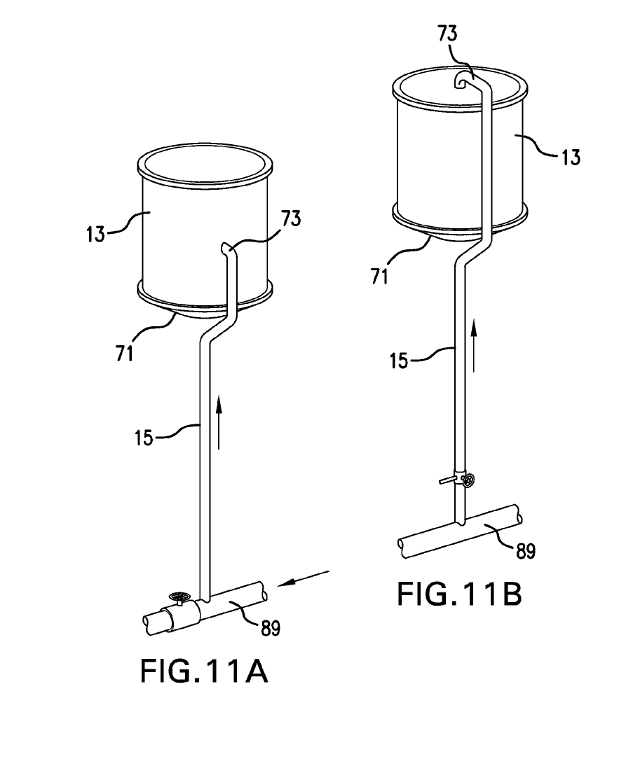

Referring to FIGS. 1 and 11, the gas exchange tank (13) has an outlet for the elimination of gas. Typically, this outlet for the elimination of gas is positioned above the fluid fill level (75) along a wall or top of the gas exchange tank (13). During operation of the bioreactor (1), gas, especially diatomic oxygen, flows out of the gas exchange tank (13) through outlet for the elimination of gas (77). The outflow is driven by pressure that builds up in the gas exchange tank (13). In a preferred embodiment of a bioreactor (1) or bioreactor farm (discussed below) having a series of bioreactor cores (10), there is a manifold (79) which connects to outlet for the elimination of gas (77) from the bioreactor (1) or bioreactor core (10) in a farm (2) such that oxygen is collected. The manifold (79) has a nozzle and the oxygen can be potted or ported to be used as a combustion enhancer or for other uses.

Referring to FIGS. 3 and 11, the central feed pipe (15) is an elongated conduit that is a fluid communication between the outlet side of a pump (21) (discussed below) and the slurry entry inlet (73) of the gas exchange tank (13). Typically, the central feed pipe (15) has vertical riser section and runs in the center of the stack (7) along its vertical axis. In a preferred embodiment, a sparge (11) for introducing a gas is in communication with the central feed pipe (15). During operation of the bioreactor (1) or bioreactor farm (2), slurry recycles and becomes rich in dissolved diatomic oxygen. This dissolved oxygen impedes the growth of algae and is a desirable product. The sparge (11) facilitates liberation of the dissolved oxygen. A gas, usually air, is injected into the central feed pipe (15) through this sparge (11) so as to generate bubbles. These bubbles are believed to be nucleation centers for the release of dissolved form the slurry for ultimate recovery by way of the gas exchange tank (13).

In a preferred embodiment, the sparge (11) introduces a robust froth of microbubbles in the central feed pipe (15) having significant surface area to facilitate release of dissolved diatomic oxygen. In a more preferred embodiment, the sparge has porous element made from sintered stainless steel or air stone and preferably from sintered stainless steel. Typically, an air compressor provides the air (or other gas) which enter through sparge (11) and travels up the central feed pipe (15) so as to break oxygen molecules from the slurry as it enters the gas exchange tank (13).

Preferably, the air compressor is a rotary screw air compressor for this is an efficient air compressor.

Referring to FIGS. 6 and 12, the pump (21) has an inlet side and an outlet side with the inlet side in fluid communication with the settling tank (19) (discussed below) and the outlet side with the central feed pipe (15). Preferably, the pump generates a pulsing fluid flow so as to enhancing splashing in the gas exchange tank (13) as discussed above. In the embodiments of this invention that are a bioreactor farm (2), there is no significant back up of slurry flow so that this pulsing slurry flow translates to each bioreactor core (10) in a bioreactor farm (2). Most preferably, the pump (21) is a diaphragm pump which pulses fluid. This type of pump (21) is more restricted than impeller type pump (21) and results in greater residency time of carbon dioxide and nitrogen oxides in the tube (5); namely, a fifteen minute residency time is achievable.

Referring to FIG. 6, there can be a nutrient tank (91) for nutrients in fluid communication through a pipe (93) with the gas exchange tank (13) of a bioreactor (1) or first bioreactor core (10) of a bioreactor farm (2).

Continuing to refer to FIGS. 6 and 12, there is a settling tank (19). This settling tank (19) serves the functions of being a receiving and mixing tank for an inoculation of algae, nutrients and water and a reservoir for recovering slurry exiting a bioreactor (1) or bioreactor farm (2). A return pipe (23) makes a fluid communication between the lower opening (67) of the tube (5) of a bioreactor (1) or the lower opening (67) of the tube (5) of the final bioreactor core (10) of a bioreactor farm (2). This closes the system and entry in or out of the system is controlled as described above. Thus, an alien microorganism is impeded from entering the bioreactor (1) or bioreactor farm (2).

Referring to FIGS. 1 and 2, optionally, the tube (5) of a bioreactor (1) has one or more means for accessing fluid for analysis. One structure of the means for accessing fluid for analysis is an outlet valve (81) through which liquid samples are taken. Another structure is a port that is fluid tight for the mounting and insertion of probes into the tube (5) for the continuous measurement of a parameter.

Referring to FIGS. 6 and 12, optionally and preferably, there is means for harvesting (95) in communication with the tube (5), return pipe (23) or settling tank (19) for harvesting microorganism; especially algae. Preferably, the drain (83) is in fluid communication with the return pipe (23). Structures for the means for harvesting (95) are a tap, valve, quick release, Y-connector, T-connector, shunt and combinations of the foregoing.

Referring to FIGS. 1 and 7, optionally and preferably, there is a greenhouse frame (85) for supporting a greenhouse structure so as to enclose a bioreactor (1) or bioreactor farm (2) during winter and/or periods of inclement weather.

Referring to FIGS. 1 and 7, optionally and preferably, there is an all weather enclosure box (87) with electronics. The enclosure box (87) houses electronics that connect to sensors as well as to a central processor for a bioreactor (1) or bioreactor core (10). In a bioreactor farm (2) each bioreactor core (10) optionally and preferably is automated and it works in tandem with other bioreactor cores (10). This automated feature increases the reliability of operation of each bioreactor core (10) and the combined harvesting cycle of the bioreactor farm (2).

Referring to FIGS. 1 and 12, depicted is a bioreactor farm having a plurality bioreactor cores (10). Preferably, there are between about three to about ten bioreactor cores (10) in the bioreactor farm (2) and most preferably there are five. The bioreactor cores (10) are conjoined or connected together in a successive series of bioreactor cores (10). The conjoined in series is accomplished by a pipe extending from the lower opening (67) of the tube (5) of preceding bioreactor core (10) making a fluid communication with the central feed pipe (15) of a succeeding bioreactor core (10). Slurry exits a preceding bioreactor core (10) with sufficient hydraulic force to climb the central feed pipe (15) of a succeeding bioreactor core (10) and enter the gas exchange tank (13) of that bioreactor core (10).

Referring to FIG. 11A, there is an illustration of a gas elimination outlet (77) and manifold (79) in connection with a gas exchange tank (13) and FIG. 1 IB is a perspective view of a center feed pipe (15) in connection with a gas exchange tank (13).

Optionally, there can be secondary piping and valves in connection with the main center feeds (15) and tube (5) so that a bioreactor core (10) in bioreactor farm (2) can be isolated for cleaning where the pump is operated at high capacity to flush out the bioreactor core (10) and farm (2).

Continuing to refer to FIG. 12, the bioreactor farm (2) has a settling tank (19). There is a pump (21) having an inlet and an outlet side with the inlet side in fluid communication with the settling tank (19) and the outlet side in fluid communication with the central feed pipe (15) of a first bioreactor core (10).

Referring to FIGS. 1 and 2, the first bioreactor core (10) of a bioreactor farm (2) is mounted on legs (89) or suspended by cables (49) at a given elevation and has a gas exchange tank (13). In the series, each subsequent bioreactor core (10) is at lower elevation. The difference in elevation should be sufficiently great that the fluid level (75) in the gas exchange tank (13) of a subsequent bioreactor core (10) is below the fluid in the gas exchange tank of a preceding bioreactor (10). The elevation of a succeeding bioreactor core (10) decreases relative to preceding bioreactor core (10) by between about 0.5 feet to about 6 feet and most preferably, there is an about one foot difference or decline in elevation. This facilitates slurry exiting a preceding bioreactor core (10), climbing the central feed pipe (15) of a succeeding bioreactor core (10) and entering the gas exchange tank (13) of that bioreactor core (10).

Continuing to refer to FIG. 12, off of the lower opening (67) of the tube (5) of the final bioreactor core (10), a return pipe (23) is in fluid communication with a settling tank (19). Thus, there is closed system with controlled entry and exit of material from the system. Thus, an alien microorganism is impeded from entering the bioreactor farm (2).

The bioreactor farm (2) can have the same optional equipment as described above for a bioreactor.

### INDUSTRIAL APPLICABILITY

The method of operating a bioreactor (1) and/or bioreactor farm (2) is a multi-step process. Water is introduced into the settling tank (19). During the operation of the bioreactor (1) extra water may be needed. A microorganism is introduced into the settling tank (19). Less preferably, the microorganism strain could be introduced through the tube (5) or in to the gas exchange tank (13).

The microorganism can be a natural microorganism or genetically engineered microorganism. Preferably, the microorganism is algae. Strains of algae have been identified as suitable for metabolizing carbon dioxide and/or nitrogen oxides and/or for the production of combustible oil extraction. Some of these strains have the characteristic of high lipid content, high protein content and/or high starch content.

Examples of such strains are found as members of the following algae genera: Anabaena, Botryococcus, Chlorella, Dunaliella, Euglena, Haematococcus, Nannochloris, Nannochloropsis, Neochlo\acute{η}s, Nostoc, Phaeodactylum, Prymnesium, Scenedesmus, Spirulina, Synecoccus and Tetrαselmis. Among these, the presently preferred strains for lipid extraction are found as members of the following genera: Botryococcus, Chlorella, Dunaliella, Nannochloris, Nannochloropsis, Neochloris, Nostoc, Phaeodactylum, Prymnesium, Scenedesmu, and Tetraselmis. Suitable bacteria may include Alcanivorax and Cycloclastiscus.

Nutrients are introduced into the settling tank (19). Preferably, the nutrients are animal manure, microbially digested cow manure, treated sewage and fertilizer. More preferred nutrients are animal manure and fertilizer. The bioreactor (1) and bioreactor farm (2) are vehicles for disposing of manure and sewage.

The pump (21) is actuated so as pump material from the settling tank (19) to the gas exchange tank (13) along with the introduction of gas into the central feed pipe (15) through the sparge (11). From the gas exchange tank (13), the slurry flows under the force of gravity through the tube (5) that makes up the stack (7). Accordingly, the tube (5) that makes up the stack becomes loaded with an aqueous mixture of microorganism (usually algae) and nutrients. Thereafter, it either flows through the return pipe (23) to the settling tank (19) or into the next reactor (10) in a series bioreactors in bioreactor farm (2) unit it exits the final bioreactor (1) and is brought back to the settling tank (19) via the return pipe (23).

Gaseous carbon dioxide, gaseous nitrogen oxides, an effluent containing carbon dioxide and/or an effluent containing nitrogen oxides and/or other pullatants are introduce into the sparge or inlet (11) in communication with the tube (5). Carbon dioxide is regarded as a substance required for efficient growth of algae. In one embodiment, carbon dioxide is supplied to the system from tanks where this commercially available substance is held, normally in solid form, known as dry ice. It is believed that nitrogen oxide dissolves in the slurry and is taken up and metabolized by the microorganism which may be an algae. Thus, carbon dioxide and nitrogen oxides are sequestered. Nitrogen oxides are metabolized by certain strains of microorganisms into biomass. Likewise, other pollutants oxides are metabolized by certain strains of microorganisms into biomass.

In accordance with the preferred method of operating a bioreactor (1) or a bioreactor core (10) of bioreactor farm (2), carbon dioxide is pumped from its storage tank to adjust the alkalinity of the content of the tube (5) to between about pH 6.0 to pH 7.5 and preferably, pH 6.5. The amount of nutrients added to the bioreactor (1) or series of bioreactor cores (10) in a bioreactor farm (2) can be adjusted from time-to-time to obtain a desired ratio of elements in the contents of the tube (5) that makes up the stack (7) In one embodiment of this method, it is a goal that during the operation of the bioreactor (1) or series of bioreactor cores (10) in a bioreactor farm (2) to reach a level where the ratio of carbon, to nitrogen to phosphorous is about 106:16:1 (106 C, 16 N and 1 P).

In an alternative embodiment of the present invention, the bioreactor or bioreactor farm is harvested through a means for harvesting (95) in communication with the settling tank (19) to generate feedstock rich in microorganism (usually algae) to be used as a feedstock for making biofuel and biomass. The means for harvesting has structures such as a pipe, a tap, a T-connector, a valve and/or a quick release. The harvested slurry can be dewatered and pressed to produce raw combustible oil and biomass. The algae are normally harvested from the bioreactor (1) or series of bioreactor cores (10) in a bioreactor farm (2) when the mass of live algae becomes approximately thirty percent (30%) of the total weight in the tube (5).

The previously described versions of the present invention have many advantages. One advantage is the sequestration of carbon dioxide and nitrogen oxides from industry waste and converting it to algae mass/biomass. This is considered to have a significant beneficial effect for the environment and is an important advantage of the present invention. Another advantage is the collection of oxygen which is usable for the enhancement of combustion. Another advantage of the present invention is that it is employs gravity to move material so as to energy efficient, not require extensive use of pumps and mechanical and thereby be less prone to breaking with concomitant down time and repair costs. Another advantage is that the bioreactor is easy to assemble from kits of frame parts, straight lengths of tube (5), elbows and other components.

### EXAMPLES

The following examples further describe and demonstrate embodiments within the scope of the present invention. The examples are given solely for the purpose of illustration and are not to be construed as limitations or restrictions of the present invention, as persons skilled in the art will quickly realize many variations thereof are possible that are all within the spirit and scope of the invention.

### Example 1

Example 2 is an example of a bioreactor (1). Overall, the bioreactor has a truncated pyramid like shape. At the bottom, there is an approximately ten feet by ten feet by 10 feet (10′×10′) square base that comprises 100 square feet. The bioreactor (1) is approximately nine feet seven inches (9′ 7″) high. There is an approximate two feet by two feet (2′×2′) square shape on top.

### Example 2

Example 2 is and example of a bioreactor farm having five bioreactor cores (10). The bioreactor cores (10) have over about 3,300 feet of four inch (4″) clear polycarbonate tube (5). Each bioreactor (1) occupied 950 square feet. It is estimated that 45 bioreactor cores (10) could be placed on one acre.

### Example 3

Example 3 is an example of the residency time of carbon dioxide in a bioreactor (1). Carbon dioxide was introduced into the tube (5) of a bioreactor and there was residency time of over 10 minutes.

Although the present invention has been described in considerable detail with reference to certain preferred versions thereof, other versions are possible with substituted, varied and/or modified materials and steps are employed. For example, a kit of frame parts, straight lengths of tube (5), elbows and other components to assemble a bioreactor. These other versions do not depart from the invention. Therefore, the spirit and scope of the appended claims should not be limited to the description of the preferred versions contained herein.

What is claimed is:

1. A bioreactor farm comprising:
at least first and second bioreactor cores connected in series, each of the first and second bioreactor cores comprising:
a support;
a tube which at a minimum partially passes light there through, is mounted on the support and includes an upper opening, wherein at last a portion of the tube continuously runs and curls with declination about a vertical axis to form a stack of levels with each level encompassing about 360 degrees around the vertical axis where the radial distance between the tube and the vertical axis indexes within the stack so as to enhance the tube's exposure to light emanating from above the stack relative to the tube being vertically aligned at a constant radial distance from the axis within the stack; the tube also comprising a lower opening;
wherein the first bioreactor core is supported at an elevation higher than an elevation of the second bioreactor core.

2. The bioreactor farm according to claim 1 additionally comprising a gas exchange tank that has a mounting to the support at a position that is generally above the stack, is in fluid communication with the upper opening of the tube, has a slurry entry inlet and has an outlet for the elimination of gas.

3. The bioreactor farm according to claim 2 additionally comprising a central feed pipe in fluid communication with the slurry entry inlet of the gas exchange tank.

4. The bioreactor farm according to claim 1, wherein a difference in elevation between the first and second bioreactor cores is sufficient to cause slurry to move down the tube of the first reactor core and up to the upper opening of the tube of the second bioreactor core.

5. The bioreactor farm according to claim 1 additionally comprising a first gas exchange tank supported at a position that is generally above the tube of the first bioreactor core and a second gas exchange tank supported at a position that is generally above the tube of the second bioreactor core, the first gas exchange tank including an upper fill level that is at a vertical position that is higher than a slurry inlet of the second gas exchange tank.

6. The bioreactor farm according to claim 5, wherein the second bioreactor core is configured to prevent a level of liquid slurry in the second gas exchange tank from rising above the slurry inlet of the second gas exchange tank.

7. A bioreactor core comprising:
a first support;
a first annular tube which substantially passes light there through is mounted on the support, the tube including an upper opening and at least a portion of the tube extending downwardly from the upper opening, along an annular path and with declination about a vertical axis to form a stack of a plurality of levels that are in substantially parallel planes that are vertically spaced with a slope of declination lowering each level and in a direction from top to bottom the radial distance between the tube and the vertical axis within the stack increases; and
a first gas exchange tank supported at a position that is generally above the stack, is in fluid communication with the upper opening of the tube, the first gas exchange tank also has a first fluid fill level and a first slurry entry inlet above the first fluid fill level, the first slurry entry inlet being spaced sufficiently above the first fluid fill level such that as slurry entered into the first gas exchange tank through the first slurry entry inlet, the slurry splashes down onto an upper surface of the fluid at the first fluid fill level, thereby enhancing the release of gas from the slurry.

8. The bioreactor core according to claim 7 additionally comprising a central feed pipe in fluid communication with the outlet side of a pump and the slurry entry inlet of the gas exchange tank and a sparge in communication with the central feed pipe for introducing a gas.

9. The bioreactor core according to claim 7, wherein the diameter of the tube is between about 3 inches to about 5 inches.

10. The bioreactor core according to claim 7, wherein the stack has a vertical height of about 10 feet.

11. The bioreactor core according to claim 7, in combination with a second bioreactor core connected to an outlet of the first annular tube, the second bioreactor core including a second gas exchange tank, the second gas exchange tank being supported at a position that is generally above a second stack of the second bioreactor core, the second gas exchange tank comprising a second fluid fill level and a second slurry entry inlet above the second fluid fill level, the second slurry entry inlet being spaced sufficiently above the second fluid fill level such that as slurry entered into the second gas exchange tank through the second slurry entry inlet, the slurry splashes down onto an upper surface of the fluid at the second fluid fill level, thereby enhancing the release of gas from the slurry.

12. The bioreactor core according to claim 11, wherein the second fluid fill level is at a vertical height that is lower than the first fluid fill level.

13. The bioreactor core according to claim 7, wherein the first bioreactor core is configured to prevent a level of liquid slurry in the first gas exchange tank from rising above the first fluid fill level.

14. The bioreactor core according to claim 11, wherein the first bioreactor core is configured to prevent a level of liquid slurry in the first gas exchange tank from rising above the first fluid fill level, and wherein the second bioreactor core is configured to prevent a level of liquid slurry in the second gas exchange tank from rising above the second fluid fill level.
